(19) United States
(12) Patent Application Publication
(10) Pub. No.: US 2015/0304553 A1
(43) Pub. Date: Oct. 22, 2015

(54) METHOD FOR DETECTING DROPS OF A LIQUID ON A SURFACE OF A TEST SPECIMEN

(71) Applicant: TEXTEST AG, Schwerzenbach (CH)

(72) Inventor: Hugo Knecht, Schofflisdorf ZH (CH)

(73) Assignee: TEXTEST AG, Schwerzenbach (CH)

(21) Appl. No.: 14/435,086

(22) PCT Filed: Oct. 15, 2012
(86) PCT No.: PCT/EP2012/070428

§ 371 Date: Apr. 10, 2015

(51) Int. Cl. H04N 5/232; G01N 33/36; H04N 5/225; G01N 15/14

(52) U.S. Cl. CPC H04N 5/23229; G01N 15/1434; G01N 33/36; H04N 5/2256; H04N 5/2252

(57) ABSTRACT

The invention relates to a method for detecting drops of a liquid on a surface of a test specimen (16) originating from a liquid applied under pressure to an opposite surface of the test specimen during at least one test cycle comprising at least the steps of: a) monitoring the surface of the test specimen (16) with the aid of a digital camera (12); b) recording data, wherein the data are at least one image or at least one image and additional data information, at incidence of an event during a test process wherein the event is at least one of: start of the test cycle, incidence of at least one drop on the surface of the test specimen, end of the test cycle, manually initiated image recording, alarm situation.

The invention relates to a method for detecting drops of a liquid on a surface of a test specimen according to the preamble of claim 1. Furthermore, the invention relates to a drop detection device for a hydrostatic head tester arrangement and to a hydrostatic head tester arrangement.

Typically, a visual test cycle for detecting at least one drop of a liquid on a surface of the test specimen lasts several minutes, e.g. 15 minutes or more, while most of the time during testing no event happens, i.e. an event like incidence of at least one drop of a liquid on the surface of the test specimen. The period in which no event happens is fatiguing for the person carrying out testing. Nevertheless, such testing requires a high level of concentration in order not to miss the point of time where a drop or drops of liquid appear on the surface of the test specimen. It is known to apply a camera for monitoring the incidence of appearance of drops of liquid on the surface of the test specimen in order to support a user of a hydrostatic head tester arrangement to identify an event like incidence of drops of liquid on the surface of the test specimen.

An object of the present invention is to provide an improved method for detecting drops of a liquid on a surface of a test specimen, e.g. as a coated or uncoated fabric or as non-wovens or as a plastic foil, wherein this method allows to determine the exact point of time, the exact test pressure, the exact position and the shape and size of at least one drop of liquid on the surface of the test specimen.

The object is solved by the features of the characterizing part of claim 1. Embodiments are given in dependent claims.

A method for detecting drops of a liquid on a surface of a test specimen originating from a liquid applied under pressure to an opposite surface of the test specimen during at least one test cycle according to the present invention comprises at least the steps of:
a) monitoring a surface of the test specimen with the aid of a digital camera;
b) recording monitoring data provided by the digital camera, wherein the monitoring data are at least one image data or at least one image data and additional data information, at incidence of an event during a test cycle wherein the event is at least one of: start of a test cycle, incidence of at least one drop on the surface of the test specimen, end of the test cycle, manually initiated image recording, alarm situation.

Thereby, any possible event arising during testing can be detected, e.g. the exact point of time, the exact test pressure, the exact position and the shape and size of the at least one drop of liquid can be determined when the liquid penetrates through the test specimen. An alarm situation can arise e.g. when the test specimen tears apart due to sensitive material characteristics, e.g. when a test header is pressed against the test specimen in applying a too high clamping force and the material is damaged therewith. Another alarm situation can arise e.g. when the test pressure exceeds a predetermined value.

In one embodiment, the method further comprises the step of:
c) protecting the surface of the test specimen against extraneous light;
d) illuminating the flat or bulbous surface of the test specimen only indirectly;
   wherein steps c) to d) are carried out during step a).

Thereby, misinterpretation of an event like the incidence of a drop of liquid can be prevented since no extraneous light can imply the presence of at least one drop of liquid in misinterpreting a point of light as being a drop of liquid. Such false drop incidences can arise on flat surfaces as well as when the test specimen bulges due to material characteristics. In case, the surface is illuminated by direct light points this can be perceived as drops of a liquid and thus, misinterpreted as being drops of liquid.

In a further embodiment, the method further comprises the step of:
e) selecting determined monitoring data of the monitoring data recorded.

Thereby, a specific event of the test cycle can easily be traced even during long lasting test cycles.

In a further embodiment, the method further comprises the step of:
f) displaying the image recorded simultaneously or delayed to recording.

Thereby, the whole test cycle can be followed while the test process is running without need for direct visual inspection of the test specimen. Thus, the test specimen can be kept in a casing excluded from extraneous light so that no false event is recorded. It is conceivable to display an individual image of an image sequence selectable by a push-button or to select an image sequence in carrying out a search run in paging forwards or backwards in slow motion technique or in accelerated technique.

In a further embodiment, the method further comprises the step of:
g) transmitting and storing the data into an electronic storage device.

Thereby, the outcome of the test cycle is available for quality management purposes for example, e.g. safeguarding the traceability of tested test specimen later on, if need be. It is conceivable to record the whole image sequence of the test cycle but only to store the images straight before, during and straight after the event has happened. It is also conceivable to store the data of the images in a memory device of the hydrostatic head tester arrangement or in a memory device located remote from the hydrostatic head tester arrangement or to transmit the data recorded via a network to a personal computer or a central database for storage.

In one embodiment of the method, the image is linked to an actual test time and/or to an actual test pressure.

In one embodiment, additional data information is at least one of date, time, material number, testing parameter, testing time, test pressure, number of drops counted.

In one embodiment of the method, the test specimen is a coated or uncoated fabric or non-wovens or a plastic foil.

Another aspect of the invention relates to a drop detection device for implementing the method according to the present invention for a hydrostatic head tester arrangement comprises a housing, a light source and an objective lens of a digital camera being arranged inside the housing opposite of a detection opening of the housing. A ring-shaped diaphragm is arranged in a region between the light source and the detection opening.

Thereby, any event arising during testing can be detected, e.g. the exact point of time, the test pressure, the exact position, as well as the size and the shape of the drop of a liquid can be identified when the liquid penetrates through the surface of the test specimen. Furthermore, false interpretation of a drop incidence event can be prevented since no direct light can imply the presence of a drop of a liquid in misinterpreting a point of light as being a drop.

In one embodiment, a further ring-shaped diaphragm encloses the objective lens.

In one embodiment, a recording unit and/or an image displaying unit is operationally connected to the digital camera.

In one embodiment, a transparent plate, preferably a glass plate, is arranged underneath the objective lens.

In one embodiment, a diffuser is arranged in a region between the first diaphragm and the second diaphragm.

In one embodiment, the light source is an LED arrangement, preferably a LED strip.

In one embodiment, the casing comprises an inspection opening having a removable cover. Thereby, the test specimen can be inspected at any time needed, e.g. at incidence of an alarm event to check whether the test specimen is mounted correctly to the test specimen holder.

A further aspect of the invention relates to a hydrostatic head tester arrangement comprising a test pressure generating container; a clamping device with two clamping rings and a power transmission device for clamping the clamping rings against each other. Further, a drop detection device according to the present invention is operationally connected to the head tester arrangement. Thereby, any event arising during testing can be detected, e.g. the exact point of time and the exact position, the test pressure, the shape and size of the drop of a liquid can be identified when the liquid penetrates through the surface of the test specimen.

The present invention is further explained with the aid of exemplified embodiments, which are shown in figures. There is shown in:

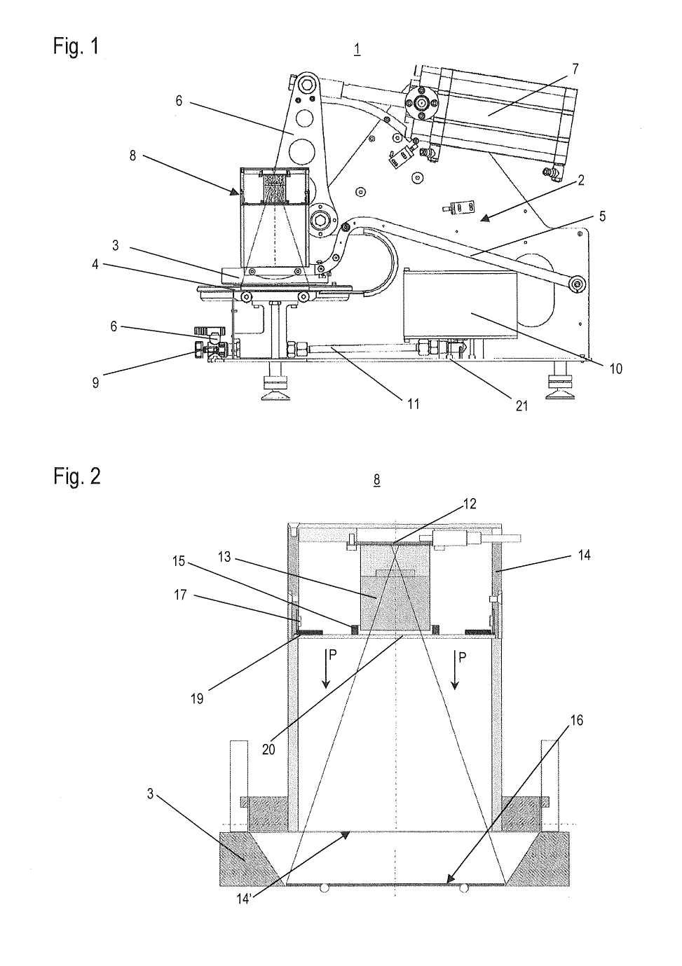

FIG. 1, schematically, a side view of a hydrostatic head tester arrangement according to the present invention is depicted; and

FIG. 2, schematically, a detection device according to the present invention for a hydrostatic head tester arrangement is depicted.

In FIG. 1, schematically, a side view of a hydrostatic head tester arrangement 1 according to the present invention is depicted. The hydrostatic head tester arrangement 1 comprises a frame 2 being mounted onto a base plate 21. A clamping device comprising an upper clamping ring 3 and a lower clamping ring 4 for clamping a test specimen (not shown in FIG. 1) with the aid of the clamping device is depicted in FIG. 1. The upper clamping ring 3 is attached to the frame 2 via a pivotable guide arm 5.

An L-shaped lever 6 transmits a predetermined contact force originating from a power transmission device 7 like e.g. a pneumatic cylinder arrangement via the upper clamping ring 3 to an upper surface of the test specimen (not shown in FIG. 1). A testing pressure is transmitted via a test pressure generating container 10 to the opposite surface, i.e. the lower surface of the test specimen. A drop detection device 8 for monitoring incidence of drops onto a surface of the test specimen can be operationally connected to the hydrostatic head tester arrangement 1. The drop detection device 8 can be arranged in a region above the upper clamping ring 3. Thereby, the drop monitoring device 8 can monitor the course of a testing cycle when the test specimen is submitted to test pressure. The lower clamping ring 4 is connected to a tank for the reception of spilled test liquid i.e. water. Further, a drain tap 9 is operationally connected to the test pressure generating container 10 via a connecting tube 11.

In FIG. 2, schematically, the drop detection device 8 according to the present invention for a hydrostatic head tester arrangement 1 is shown. The drop detection device 8 comprises a digital camera 12, wherein an objective lens 13 of the digital camera 12 is arranged inside a housing 14 of the drop detection device 8. A first ring-shaped diaphragm 15 is arranged around the objective lens 13. The diaphragm 15 serves to prevent light reflected e.g. by a protection glass 20 to enter the objective lens 13. The test specimen 16 is e.g. a plastic foil, a coated or uncoated fabric or a non-woven. A light source 17 e.g. a LED arrangement is arranged at an inner side of the cylindrical housing 14 above a second diaphragm 19. The light source 17 together with the second diaphragm 19 allows an indirect illumination of the surface of the test specimen 16 being depicted by arrows P. The light source 17 is arranged approximately at the same height as the first ring-shaped diaphragm 15. The second ring-shaped diaphragm 19 is arranged underneath the light source 17. The second ring-shaped diaphragm 19 prevents the test specimen 16 to be illuminated by direct light from the light source 17. The width of the second ring-shaped diaphragm 19 is dimensioned such that direct light originating from the light source 17 is covered or hidden with respect to a detection opening 14′ of the housing 14. Thus, the test specimen 16 is only illuminated indirectly via the light source 17. Optionally, the transparent protection glass 20 is mounted to protect the objective lens 13 against splashing water. Furthermore, it is conceivable to arrange a diffuser (not shown in FIG. 2) in a region between the first diaphragm 15 and the second diaphragm 17 such that the objective lens 13 remains free. Depending on the material characteristics of the test specimen 16, an upper surface of the test specimen is flat or bulbously. The housing 14 is arranged to the upper clamping ring 3 such that no extraneous light, e.g.

light from outside the housing 14 can penetrate into housing 14. The drop detection device 8 can be implemented in a compact way such that it easily can be incorporated into the hydrostatic head tester arrangement 1.

1. Method for detecting drops of a liquid on a surface of a test specimen (16) originating from a liquid applied under pressure to an opposite surface of the test specimen (16) during at least one test cycle comprising at least the steps of:
a) monitoring the surface of the test specimen (16) with the aid of a digital camera (12);
b) recording monitoring data provided by the digital camera (12), wherein the monitoring data are at least one image data or at least one image data and additional data information, at incidence of an event during a test process wherein the event is at least one of: start of the test cycle, incidence of at least one drop on the surface of the test specimen, end of the test cycle, manually initiated image recording, alarm situation.

2. Method according to claim 1, the method further comprises the step of:
c) protecting the surface of the test specimen (16) against extraneous light;
d) illuminating the surface of the test specimen (16) only indirectly;
wherein steps c) to d) are carried out during step a).

3. Method according to claim 1, the method further comprises the step of:
e) selecting determined monitoring data of the monitoring data recorded.

4. Method according to claim 1, the method further comprises the step of:
f) displaying the image recorded simultaneously or delayed to recording.

5. Method according to claim 1, the method further comprises the step of:
g) transmitting and storing data into an electronic storage device.

6. Method according to, wherein the image data is linked to an actual test time and/or to an actual test pressure.

7. Method according to claim 1, wherein additional data information is at least one of date, time, material number, testing parameter, testing time, test pressure, number of drops counted.

8. Method according to claim 1, wherein the test specimen is a coated or uncoated fabric or non-wovens or a plastic foil.

9. Drop detection device (8) for implementing the method according to claim 1 for a hydrostatic head tester arrangement (1) comprising a housing (14), a light source (17) and an objective lens (13) of a digital camera (12) being arranged inside the housing (14) opposite of a detection opening (14′) of the housing (14), a ring-shaped diaphragm (19) being arranged in a region between the light source (17) and the detection opening (14′).

10. Drop detection device (8) according to claim 9, characterized in that a further ring-shaped diaphragm (15) encloses the objective lens (13).

11. Drop detection device (8) according to claim 9, characterized in that a recording unit and/or an image displaying unit is operationally connected to the digital camera (12).

12. Drop detection device (8) according to claim 9, characterized in that a transparent plate (20), preferably a glass plate, is arranged underneath the objective lens (13).

13. Drop detection device (8) according to claim 9, characterized in that a diffuser is arranged in a region between the first diaphragm (15) and the second diaphragm (19).

14. Drop detection device according to claim 9, characterized in that the light source (17) is a LED arrangement, preferably a LED strip.

15. Drop detection device (8) according to claim 9, characterized in that the housing (14) comprises an inspection opening having a removable cover.

16. Hydrostatic head tester arrangement (1) comprising a test pressure generating container (10); a clamping device with two clamping rings (3; 4) and a power transmission device (7) for clamping the clamping rings (3; 4) against each other, characterized in that a drop detection device (8) according to claim 9 is operationally connected to the head tester arrangement (1).
